(19) United States
(12) Patent Application Publication
(10) Pub. No.: US 2025/0127222 A1
(43) Pub. Date: Apr. 24, 2025

(54) AEROSOL DELIVERY ARTICLE WITH AN INFRARED HEATER

(71) Applicant: Imperial Tobacco Limited, Bristol (GB)

(72) Inventors: Mohammed Benyezzar, Liverpool Merseyside (GB); David Jones, Liverpool Merseyside (GB); Edward Ross Shenton, Liverpool Merseyside (GB)

(21) Appl. No.: 18/694,406

(22) PCT Filed: Sep. 8, 2022
(86) PCT No.: PCT/EP2022/074946

§ 371 Date: Mar. 21, 2024

(30) Foreign Application Priority Data
Sep. 27, 2021 (EP) 21199100.5

(51) Int. Cl. A24F 40/46; A24F 40/10; A24F 40/20; A24F 40/42

(52) U.S. Cl. CPC A24F 40/46; A24F 40/10; A24F 40/20; A24F 40/42

(57) ABSTRACT

The present disclosure provides an aerosol forming article comprising an aerosol forming article comprising a cavity for receiving an aerosol former, an infrared heater in thermal communication with the cavity, where the infrared heater comprises at least one heating element in a halogen-filled enclosure and where the enclosure at least partly circumscribes the cavity. There is also provided an aerosol delivery article comprising: a cavity for receiving an aerosol former, the cavity having a perimeter, and an IR heater comprising at least one heating element for heating the aerosol former. A major portion of the perimeter of the cavity faces away from the heating element(s).

### TECHNICAL FIELD

The present invention relates to an aerosol delivery article and particularly, although not exclusively, to an aerosol delivery article which may be an aerosol delivery device or an aerosol delivery component of an aerosol delivery system.

### BACKGROUND

The smoking of tobacco is generally considered to expose a smoker to potentially harmful substances. It is generally thought that a significant amount of the potentially harmful substances are generated through the heat caused by the burning and/or combustion of the tobacco and the constituents of the burnt tobacco in the tobacco smoke itself.

Conventional combustible smoking articles, such as cigarettes, typically comprise a cylindrical rod of tobacco comprising shreds of tobacco which is surrounded by a wrapper, and usually also a cylindrical filter axially aligned in an abutting relationship with the wrapped tobacco rod. The filter typically comprises a filtration material which is circumscribed by a plug wrap. The wrapped tobacco rod and the filter are joined together by a wrapped band of tipping paper that circumscribes the entire length of the filter and an adjacent portion of the wrapped tobacco rod. A conventional cigarette of this type is used by lighting the end opposite to the filter, and burning the tobacco rod. The smoker receives mainstream smoke into their mouth by drawing on the mouth end or filter end of the cigarette.

Combustion of organic material such as tobacco is known to produce tar and other potentially harmful by-products. There have been proposed various aerosol delivery systems (or “substitute smoking systems”) in order to avoid the smoking of tobacco.

Such aerosol delivery systems can form part of nicotine replacement therapies aimed at people who wish to stop smoking and overcome a dependence on nicotine.

Aerosol delivery systems include electronic systems that permit a user to simulate the act of smoking by producing an aerosol (also referred to as a “vapour”) that is drawn into the lungs through the mouth (inhaled) and then exhaled. The inhaled aerosol typically bears nicotine and/or flavourings without, or with fewer of, the odour and health risks associated with traditional smoking.

In general, aerosol delivery systems are intended to provide a substitute for the rituals of smoking, whilst providing the user with a similar experience and satisfaction to those experienced with traditional smoking and with combustible tobacco products. Some aerosol delivery systems use aerosol delivery components (also referred to as a “consumables”) that are designed to resemble a traditional cigarette and are cylindrical in form with a mouthpiece at one end.

The use of aerosol delivery systems has grown rapidly in the past few years as an aid to assist habitual smokers wishing to quit tobacco smoking.

There are a number of different categories of aerosol delivery systems, each utilising a different aerosol delivery approach.

One approach for an aerosol delivery system is the so-called Heated Tobacco (“HT”) approach in which tobacco (rather than an “e-liquid”) is heated or warmed to release vapour. HT is also known as “heat not burn” (“HNB”). The tobacco may be leaf tobacco or reconstituted tobacco. The vapour may contain nicotine and/or flavourings. In the HT approach the intention is that the tobacco is heated but not burned, i.e. the tobacco does not undergo combustion.

A typical HT aerosol delivery system may include a device and a consumable. The consumable may include the tobacco material. The device and consumable may be configured to be physically coupled together. In use, heat may be imparted to the tobacco material by a heater of the device, wherein airflow through the tobacco material causes components in the tobacco material to be released as vapour. A vapour may also be formed from a carrier in the tobacco material (this carrier may for example include propylene glycol and/or vegetable glycerine) and additionally volatile compounds released from the tobacco. The released vapour may be entrained in the airflow drawn through the tobacco.

As the vapour passes through the consumable (entrained in the airflow) from the location of vaporisation to an outlet of the consumable (e.g. a mouthpiece), the vapour cools and condenses to form an aerosol for inhalation by the user. The aerosol will normally contain the volatile compounds.

In HT aerosol delivery systems, heating as opposed to burning the tobacco material is believed to cause fewer, or smaller quantities, of the more harmful compounds ordinarily produced during smoking. Consequently, the HT approach may reduce the odour and/or health risks that can arise through the burning, combustion and pyrolytic degradation of tobacco.

Another approach for an aerosol delivery system is the so-called “vaping” approach, in which a vaporisable liquid, typically referred to (and referred to herein) as “e-liquid”, is heated by a heating device to produce an aerosol vapour which is inhaled by a user. The e-liquid typically includes a base liquid as well as nicotine and/or flavourings. The resulting vapour therefore also typically contains nicotine and/or flavourings. The base liquid may include propylene glycol and/or vegetable glycerine.

A typical vaping aerosol delivery system includes a device, a mouthpiece, a power source (typically a battery), a tank for containing e-liquid, as well as a heating device. In use, electrical energy is supplied from the power source to the heating device, which heats the e-liquid to produce an aerosol (or “vapour”) which is inhaled by a user through the mouthpiece.

Vaping aerosol delivery systems can be configured in a variety of ways. For example, there are “closed system” vaping aerosol delivery systems, which typically have a sealed tank and heating element. The tank is pre-filled with e-liquid and is not intended to be refilled by an end user. One subset of closed system vaping aerosol delivery systems include a device which includes the power source, wherein the device is configured to be physically and electrically coupled to a component including the tank and the heating element. In this way, when the tank of a component has been emptied, that component is disposed of. The device can be reused by connecting it to a new, replacement, component. Another subset of closed system vaping aerosol delivery systems are completely disposable, and intended for one-use only.

There are also “open system” vaping aerosol delivery systems which typically comprise a device including a tank that is configured to be refilled by a user. In this way the device can be used multiple times.

There may be a need for improved design of aerosol delivery systems, to enhance the user experience and improve the function of the aerosol delivery system.

The present disclosure has been devised in the light of the above considerations.

### SUMMARY OF THE INVENTION

At its most general, the present invention relates to an aerosol delivery article (e.g. an aerosol delivery device or an aerosol delivery component) comprising an IR heater.

According to a first aspect, there is provided an aerosol delivery article comprising:
a cavity for receiving an aerosol former; and
an infrared heater in thermal communication with the cavity, the infrared heater comprising a halogen-filled enclosure housing at least one heating element,
wherein the enclosure at least partly circumscribes the cavity.

By providing a heater with an enclosure that at least partly circumscribes the cavity for receiving the aerosol former, the enclosure can be in thermal communication with a greater surface area of the cavity. Thus, the heater can more effectively transfer heat to the cavity and therefore to the aerosol former (which may be a solid substrate or an e-liquid, for example) received in the cavity. Furthermore, by providing a halogen-filled enclosure, the halogen can enhance the transfer of heat from the heating element throughout the cavity (e.g. by radiation), as well as equalise the heat throughout the cavity. Thus, the heater can be more efficient at transferring heat to the aerosol former received in the cavity.

Optional features will now be set out. These are applicable singly or in any combination with any aspect.

In some embodiments, the cavity is at least partly defined by the enclosure.

The cavity may be an elongate cavity. The cavity may have a substantially circular transverse-profile perpendicular to the longitudinal axis of the elongate cavity i.e. the cavity may be a substantially cylindrical cavity.

In some embodiments, the cavity is defined by a chamber and the enclosure at least partly circumscribes the chamber. The chamber may be formed of a thermally conductive material. There may be direct contact between the enclosure and the chamber.

The chamber may be an elongate chamber. The chamber may have a substantially circular transverse-profile perpendicular to the longitudinal axis of the elongate chamber i.e. the chamber may be a substantially cylindrical chamber.

In some embodiments, the enclosure may comprise an outer wall, the outer wall having an indented portion, the indented portion at least partly circumscribing the cavity e.g. at least partly circumscribing the chamber. Thus the indented portion may at least partly define the cavity or may be in contact with the chamber defining the cavity.

The indented portion may define an arc projecting inwardly into the enclosure. The arc may have an arc angle equal to or greater than 180 degrees. In some embodiments, the outer wall not forming the indented portion may also form an arc e.g. an arc having a greater arc radius than the indented portion. In these embodiments, the enclosure may have a crescent-shaped transverse profile i.e. a crescent-shaped profile perpendicular to a longitudinal axis of the enclosure. In other embodiments, the outer wall not forming the indented portion may define three sides of a quadrilateral such as a square or rectangle (e.g. with the fourth side being formed by the indented portion).

In some embodiments, the enclosure completely circumscribes the cavity e.g. the enclosure completely circumscribes the chamber. For example, the indented portion may form an arc of substantially 360 degrees such that it completely circumscribes the cavity.

In some embodiments, the enclosure may comprise an outer wall and an inner wall, the outer wall surrounding the inner wall with the enclosure defined therebetween. In these embodiments, the inner wall may fully circumscribe the cavity/chamber. For example, the inner wall may define the cavity or the inner wall may be in contact with the chamber defining the cavity. In these embodiments, the outer wall (and thus the enclosure) may have a transverse profile (i.e. a profile perpendicular to the longitudinal axis of the cavity/chamber) that is a quadrilateral shape e.g. a substantially rectangular shape. The inner wall may be a circular/circumferential wall (enclosing the cavity/chamber).

The axial centre of the profile defined by the outer wall may be laterally offset from the axial centre of the cavity/chamber.

The enclosure (e.g. at least the indented portion of the outer wall or the inner wall) may be formed of thermally conductive material (e.g. glass, lime glass, Pyrex, silica) to allow heat transfer from the IR heater (e.g. the heating element) through the enclosure and to the aerosol former in the cavity.

The at least one heating element may be a filament, a strip, a heating electrode or any other suitable heating element for generating IR radiation.

The/each heating element may be elongate so as to define a longitudinal axis, e.g. parallel to the longitudinal axis of the elongate cavity/chamber.

The cavity has a perimeter (which may be defined by the chamber). In preferred embodiments, a major portion of the perimeter (i.e. a portion subtending an angle greater than 180 degrees) faces away from the heating element(s). In other words, only a minor portion (i.e. a portion subtending an angle of less than 180 degrees) faces the heating element(s).

The major portion of the perimeter may subtend an angle of equal to or greater than 200 degrees e.g. equal to or greater than 210 degrees or 250 degrees. The major portion may subtend an angle of equal to or greater than 270, 290 or 315 degrees.

The term “faces away” means that a straight line extending from the axial centre of the cavity through the perimeter does not intersect the heating element(s).

According to a second aspect, there is provided an aerosol delivery article comprising:
a cavity for receiving an aerosol former, the cavity having a perimeter,
an IR heater comprising at least one heating element for heating the aerosol former wherein a major portion of the perimeter faces away from the heating element(s).

An article where a major portion of the perimeter of the cavity (for receiving the aerosol former) faces away from the heating element(s) effectively only has a heating element provided to one lateral side of the cavity (as opposed to a heating element surrounding the cavity or heating elements provided on opposing lateral sides of the cavity). This may reduce the amount of insulation needed for the article as insulation may only be needed on the lateral side where the at least one heating element is located.

The term “faces away” means that a line extending from the axial centre of the cavity through the perimeter does not intersect the heating element(s).

In preferred embodiments, a major portion of the perimeter (i.e. a portion subtending an angle greater than 180 degrees) faces away from the heating element(s). In other words, only a minor portion (i.e. a portion subtending an angle of less than 180 degrees) faces the heating element(s).

The major portion of the perimeter may subtend an angle of equal to or greater than 200 degrees e.g. equal to or greater than 210 degrees or 250 degrees. The major portion may subtend an angle of equal to or greater than 270, 290 or 315 degrees.

The IR heater may comprise an enclosure housing the at least one heating element. The enclosure may be a gas-filled enclosure (e.g. a halogen-filled enclosure). The enclosure may at least partly circumscribe the cavity. The article may comprise a chamber defining the cavity.

The enclosure, chamber, cavity and/or the IR heater/heating filament of the article of the second aspect may otherwise be as described above with respect to the first aspect.

The aerosol delivery article of the first aspect or the second aspect may comprise a heat reflector. The reflector may at least partly circumscribe the IR heater. For example, the reflector may at least partly (e.g. fully) circumscribe the outer wall of the enclosure.

The reflector may at least partly (e.g. fully) circumscribe the cavity/chamber.

The reflector may be a coating (e.g. a metal coating).

The aerosol delivery article of the first aspect or the second aspect may comprise a heat insulator. The insulator may at least partly (e.g. fully) circumscribe the IR heater.

The insulator may at least partly (e.g. fully) circumscribe the cavity/chamber.

The insulator may circumscribe the reflector.

In some embodiments of the first and second aspects, the article is a device configured for engagement with the aerosol former in the form of an aerosol delivery component.

The device may comprise a device body (e.g. an elongate body). The IR heater and the chamber/cavity may extend within the body (e.g. parallel to the longitudinal axis of the elongate body). The IR heater may be mounted to the body.

The device body may have an opening through which the aerosol former (component) can be introduced into the chamber/cavity.

The device may comprise a power source or may be connectable to a power source (e.g. a power source separate to the device). The power source may be electrically connectable to the IR heater (e.g. to the at least one heating element). The device may comprise means (e.g. one or more heater connectors) for connecting the IR heater (e.g. the at least one heating element) with the power source. The heater connectors may be electrically connected to the heating element(s) such that electricity supplied via the heater connectors may pass to the heating element.

Altering (e.g. toggling) the electrical connection of the power source to the heating element(s) may affect a state of the IR heater. For example, toggling the electrical connection of the power source to the heating element(s) may toggle the IR heater between an on state and an off state. The power source may be a power store. For example, the power source may be a battery or rechargeable battery (e.g. a lithium ion battery).

The device may comprise an input connection (e.g. a USB port, Micro USB port, USB-C port, etc.). The input connection may be configured for connection to an external source of electrical power, such as a mains electrical supply outlet. The input connection may, in some cases, be used as a substitute for an internal power source (e.g. battery or rechargeable battery). That is, the input connection may be electrically connectable to the heater (for providing power to the heater). Hence, in some forms, the input connection may form at least part of the power source of the device.

Where the power source comprises a rechargeable power source (such as a rechargeable battery), the input connection may be used to charge and recharge the power source.

The device may comprise a user interface (UI). In some embodiments the UI may include input means to receive operative commands from the user. The input means of the UI may allow the user to control at least one aspect of the operation of the device. In some embodiments the input means may comprise a power button to switch the device between an on state and an off state.

In some embodiments the UI may additionally or alternatively comprise output means to convey information to the user. In some embodiments the output means may comprise a light to indicate a condition of the device (and/or the aerosol delivery component) to the user. The condition of the device (and/or aerosol delivery component) indicated to the user may comprise a condition indicative of the operation of the heater. For example, the condition may comprise whether the heater is in an off state or an on state. In some embodiments, the UI unit may comprise at least one of a button, a display, a touchscreen, a switch, a light, and the like. For example, the output means may comprise one or more (e.g. two, three, four, etc.) light-emitting diodes (“LEDs”) that may be located on the body of the device.

The device may further comprise a puff sensor (e.g. airflow sensor), which form part of the input means of the UI. The puff sensor may be configured to detect a user drawing on an end (i.e. a terminal (mouth) end) of the aerosol delivery component. The puff sensor may, for example, be a pressure sensor or a microphone. The puff sensor may be configured to produce a signal indicative of a puff state. The signal may be indicative of the user drawing (an aerosol from the aerosol delivery component) such that it is e.g. in the form of a binary signal. Alternatively or additionally, the signal may be indicative of a characteristic of the draw (e.g. a flow rate of the draw, length of time of the draw, etc.).

The device may comprise a controller, or may be connectable to a controller that may be configured to control at least one function of the device. The controller may comprise a microcontroller that may e.g. be mounted on a printed circuit board (PCB). The controller may also comprise a memory, e.g. non-volatile memory. The memory may include instructions, which, when implemented, may cause the controller to perform certain tasks or steps of a method. Where the device comprises an input connection, the controller may be connected to the input connection.

The controller may be configured to control the operation of the IR heater (and the heating element(s)). Thus, the controller may be configured to control vaporisation of the aerosol former forming part of the aerosol delivery component engaged with the device. The controller may be configured to control the voltage applied by power source to the heater (e.g. the heating element). For example, the controller may be configured to toggle between applying a full output voltage (of the power source) to the heater and applying no voltage to the heater. Alternatively or additionally, the control unit may implement a more complex heater control protocol.

The device may further comprise a voltage regulator to regulate the output voltage supplied by the power source to form a regulated voltage. The regulated voltage may subsequently be applied to the heater.

In some embodiments, where the device comprises a UI, the controller may be operatively connected to one or more components of the UI. The controller may be configured to receive command signals from an input means of the UI. The controller may be configured to control the heater (e.g. the heating element) in response to the command signals. For example, the controller may be configured to receive “on” and “off” command signals from the UI and, in response, may control the heater (e.g. the heating element) so as to be in a corresponding on or off state.

The controller may be configured to send output signals to a component of the UI. The UI may be configured to convey information to a user, via an output means, in response to such output signals (received from the controller). For example, where the device comprises one or more LEDs, the LEDs may be operatively connected to the controller. Hence, the controller may configured to control the illumination of the LEDs (e.g. in response to an output signal). For example, the controller may be configured to control the illumination of the LEDs according to (e.g. an on or off) state of the heater.

Where the device comprises a sensor (e.g. a puff/airflow sensor), the controller may be operatively connected to the sensor. The controller may be configured to receive a signal from the sensor (e.g. indicative of a condition of the device and/or engaged aerosol delivery component). The controller may be configured to control the IR heater (e.g. the at least one heating element), or an aspect of the output means, based on the signal from the sensor.

The device may comprise a wireless interface configured to communicate wirelessly (e.g. via Bluetooth (e.g. a Bluetooth low-energy connection) or WiFi) with an external device. Similarly, the input connection may be configured for wired connection to an external device so as to provide communication between the device and the external device.

The external device may be a mobile device. For example, the external device may be a smart phone, tablet, smart watch, or smart car. An application (e.g. app) may be installed on the external device (e.g. mobile device). The application may facilitate communication between the device and the external device via the wired or wireless connection.

The wireless or wired interface may be configured to transfer signals between the external device and the controller of the device. In this respect, the controller may control an aspect of the device in response to a signal received from an external device. Alternatively or additionally, an external device may respond to a signal received from the device (e.g. from the controller of the device).

According to a third aspect, there is provided an aerosol delivery system comprising:
a device as described above; and
an aerosol delivery component.

The aerosol delivery component comprises the aerosol former, e.g. the component may be a heated tobacco (HT) consumable component (also known as a heat-not-burn (HNB) consumable component) such that the aerosol former is a solid substrate. Thus, the system may be a heated tobacco (or heat-not-burn) system. The cavity/chamber of the device may be configured for receipt of at least a portion of the (consumable) component.

The terms “heated tobacco” and “heat-not-burn” are used interchangeably herein to describe a consumable component that is of the type that is heated rather than combusted (or are used interchangeably to describe a device for use with such a consumable component).

The IR heater of the device may be configured to heat the consumable component. In particular, the IR heater may be configured to heat and vaporise the aerosol former of the component. The aerosol former is capable of being heated to release at least one volatile compound that can form an aerosol. The aerosol former may be located at an upstream end of the component.

As used herein, the terms “upstream” and “downstream” are intended to refer to the flow direction of the vapour/aerosol i.e. with the downstream end of the component/consumable being the mouth end or outlet where the aerosol exits the consumable for inhalation by the user. The upstream end of the component is the opposing end to the downstream end.

In some embodiments of the first and second aspects, the article is a vaping component (e.g. a consumable vaping component) configured for engagement with a vaping device.

The vaping component may comprise a tank, which may define a reservoir for storage of the aerosol former. The aerosol former may be in liquid form (e.g. an e-liquid).

The vaping component may comprise a porous wick that draws e-liquid from the tank. The wick may be disposed in a vaporising chamber. The vaporising chamber may define the cavity of the vaping component for receiving the aerosol former (e.g. the e-liquid) i.e. the IR heater at least partly encloses the vaporising chamber of the vaping component. When the at least one heating element is heated (e.g. by the action of electrical current passing through the heating element), heat may be transferred from the at least one heating element through the enclosure, and to the e-liquid conveyed by the wick to the cavity/vaporising chamber. This transfer of heat may vaporise the e-liquid and the resultant vapour may be entrained in an airflow passing through the vaping component.

The vaping component may comprise means (e.g. one or more heater connectors) for connecting the IR heater (e.g. the heating element(s)) with a power source. The heater connectors may be in the form of electrically conductive elements or contacts (e.g. metal plates). The heater connectors may be electrically connected to the heating element(s), such that electricity supplied via the heater connectors may pass to the heating element(s). In other words, a voltage applied across the heater connectors may generally correspond to a voltage applied across the heating element(s) of the IR heater.

According to a fourth aspect, there is provided an aerosol delivery system comprising:
a vaping component (e.g. a consumable component) as described above; and
a vaping device.

The system is in the form of a vaping system (i.e. rather than a heated tobacco system as described above) comprising the vaping component described above. The vaping system may be configured such that the vaping component can be received and retained in a recess of the device (i.e. so as to be engaged with the device). The vaping component may be retained by way of e.g. an interference fit, screwing one onto (or onto) the other, a bayonet fitting, or by way of a snap engagement mechanism.

The vaping device may comprise a power source or may be connectable to a power, the power source being electrically connectable to the heater (e.g. the heating element) of the component.

The vaping device may comprise means for connecting the heater (e.g. the heating element) of the vaping component engaged with the vaping device. For example, the vaping device may comprise one or more device connectors for (e.g. electrically) connecting the vaping device to the IR heater of the vaping component. The vaping device connector(s) may be in the form of electrically conductive element(s) (e.g. plates) that contact when the vaping component is engaged with the vaping device.

The vaping component may be a “single-use” component. That is, upon exhausting the e-liquid in the tank, the intention may be that the user disposes of the entire component. Alternatively, the e-liquid may be the only part of the system that is truly “single-use”. For example, the tank may be refillable with e-liquid or another component of the system (internal to the vaping device or external to the vaping device e.g. a refillable cartomizer) may define a reservoir for the e-liquid.

In this embodiment, the vaping component comprises the IR heater (i.e. instead of the IR heater forming part of the device). The heater connectors of the vaping component may be disposed on an in-use device-facing surface of vaping component. The heater connector(s) may be arranged such that they contact the vaping device connector(s) when the vaping component is engaged with the vaping device. The vaping device connector(s) may be connected (e.g. electrically) to the power source (e.g. battery) of the vaping device. Thus, electricity may be supplied from the power source to the heating element(s), via in-contact heater and device connectors. In this way, the IR heater forming part of the vaping component may operate and interact with a controller.

The power source and/or controller of the vaping device may be as described above for the device having the integrated IR heater. The vaping device may further comprise an input connection, user interface, puff sensor, voltage regulator and/or wireless interface as described above for the device having an integrated IP heater.

The invention includes the combination of the aspects and preferred features described except where such a combination is clearly impermissible or expressly avoided.

The skilled person will appreciate that except where mutually exclusive, a feature or parameter described in relation to any one of the above aspects may be applied to any other aspect. Furthermore, except where mutually exclusive, any feature or parameter described herein may be applied to any aspect and/or combined with any other feature or parameter described herein.

### SUMMARY OF THE FIGURES

So that the invention may be understood, and so that further aspects and features thereof may be appreciated, embodiments illustrating the principles of the invention will now be discussed in further detail with reference to the accompanying figures, in which:

FIG. 1A is a schematic of an aerosol delivery system;

FIG. 1B is a schematic of a variation of the aerosol delivery system of FIG. 1A;

FIG. 2A is a front view of an aerosol delivery system with the consumable engaged with the device;

FIG. 2B is a front view of the aerosol delivery system of FIG. 2A with the consumable disengaged from the device;

FIG. 2C is a section view of the consumable of the aerosol delivery system of FIGS. 2A and 2B;

FIG. 2D is a detailed view of an end of the device of the aerosol delivery system of FIGS. 2A and 2B;

FIG. 3A is a front view of an aerosol delivery system with the consumable engaged with the device;

FIG. 3B is a front view of the aerosol delivery system of FIG. 3A with the consumable disengaged from the device.

FIG. 4 is a schematic diagram of a transverse section view of a first embodiment of an aerosol delivery article;

FIG. 5 is a schematic diagram of a transverse section view of a second embodiment of an aerosol delivery article; and

FIG. 6 is a schematic diagram of a transverse section view of a third embodiment of an aerosol delivery article.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

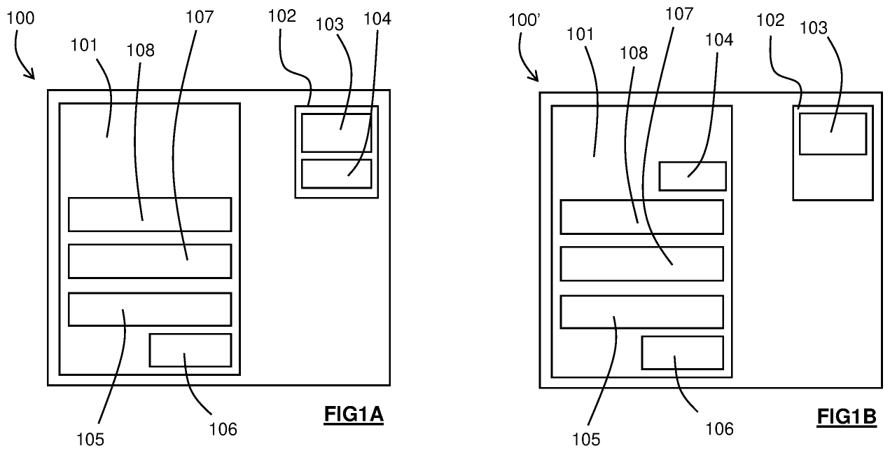

FIG. 1A is a schematic providing a general overview of an aerosol delivery system 100. The system 100 includes an aerosol delivery device 101 and an aerosol delivery component in the form of a consumable component 102, which comprises an aerosol former 103. The system is configured to vaporise volatile components in the aerosol former by heating the aerosol former 103 (so as to form a vapour/aerosol for inhalation by a user).

In the illustrated system (which may, for example, be a vaping system), the IR heater 104 forms part of the consumable component 102 and is configured to heat the aerosol former 103. In this variation, the IR heater 104 is electrically connectable to the power source 105, for example, when the consumable component 102 is engaged with the device 101. Heat from the IR heater 104 vaporises volatile components within the aerosol former 103 to produce a vapour. The vapour subsequently condenses to form an aerosol, which is ultimately inhaled by the user.

The system 100 further comprises a power source 105 that forms part of the device 101. In other embodiments the power source 105 may be external to (but connectable to) the device 101. The power source 105 is electrically connectable to the IR heater 104 such that it is able to supply power to the heater 104 (i.e. for the purpose of heating the aerosol former 103). Thus, control of the electrical connection of the power source 105 to the IR heater 104 provides control of the state of the IR heater 104. The power source 105 may be a power store, for example a battery or rechargeable battery (e.g. a lithium ion battery).

The system 100 further comprises an I/O module comprising a connector 106 (e.g. in the form of a USB port, Micro USB port, USB-C port, etc.). The connector 106 is configured for connection to an external source of electrical power, e.g. a mains electrical supply outlet. The connector 106 may be used in substitution for the power source 105. That is the connector 106 may be electrically connectable to the heater 104 so as to supply electricity to the IR heater 104. In such embodiments, the device may not include a power source, and the power source of the system may instead comprise the connector 106 and an external source of electrical power (to which the connector 106 provides electrical connection).

In some embodiments, the connector 106 may be used to charge and recharge the power source 105 where the power source 105 includes a rechargeable battery.

The system 100 also comprises a user interface (UI) 107. Although not shown, the UI 107 may include input means to receive commands from a user. The input means of the UI 107 allows the user to control at least one aspect of the operation of the system 100. The input means may, for example, be in the form of a button, touchscreen, switch, microphone, etc.

The UI 107 also comprises output means to convey information to the user. The output means may, for example, comprise lights (e.g. LEDs), a display screen, speaker, vibration generator, etc.

The system 100 further comprises a controller 108 that is configured to control at least one function of the device 101. In the illustrated embodiment, the controller 108 is a component of the device 101, but in other embodiments may be separate from (but connectable to) the device 101. The controller 108 is configured to control the operation of the IR heater 104 and, for example, may be configured to control the voltage applied from the power source 105 to the IR heater 104. The controller 108 may be configured to toggle the supply of power to the IR heater 104 between an on state, in which the full output voltage of the power source 105 is applied to the IR heater 104, and an off state, in which the no voltage is applied to the IR heater 104.

Although not shown, the system 100 may also comprise a voltage regulator to regulate the output voltage from the power source 105 to form a regulated voltage. The regulated voltage may then be applied to the IR heater 104.

In addition to being connected to the IR heater 104, the controller 108 is operatively connected to the UI 107. Thus, the controller 108 may receive an input signal from the input means of the UI 107. Similarly, the controller 108 may transmit output signals to the UI 107. In response, the output means of the UI 107 may convey information, based on the output signals, to a user. The controller also comprises a memory 109, which is a non-volatile memory. The memory 109 includes instructions, which, when implemented, cause the controller to perform certain tasks or steps of a method.

FIG. 1B is a schematic showing a variation of the system 100 of FIG. 1A (which may, for example be a HT system). In the system 100′ of FIG. 1B, the IR heater 104 forms part of the device 101, rather than the consumable component 102. In this variation, the IR heater 104 is electrically connected to the power source 105.

The systems 100, 100′ of FIGS. 1A and 1B may be implemented as one of two broad categories of system, each in accordance with the present invention: a heated tobacco (HT) system or a vaping system. A description of each category of system follows.

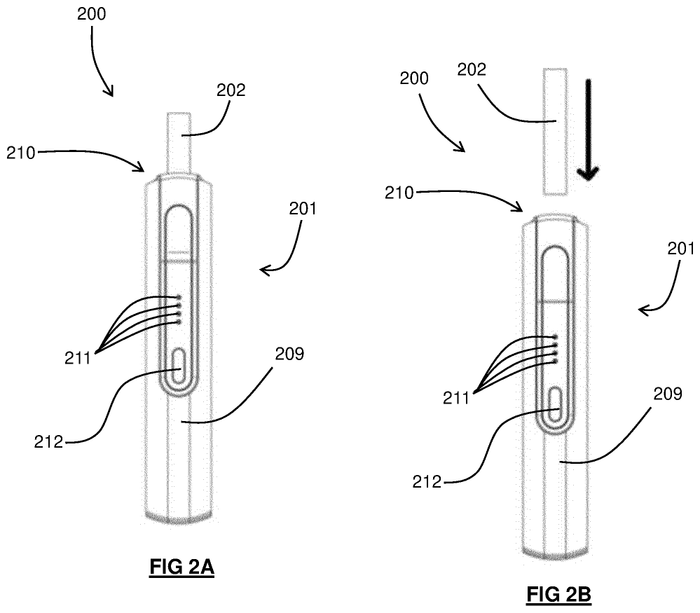

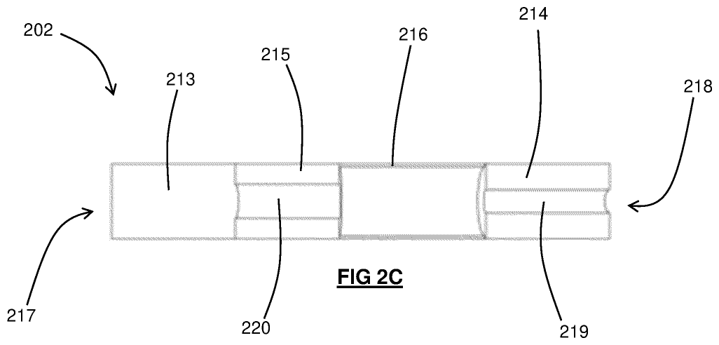

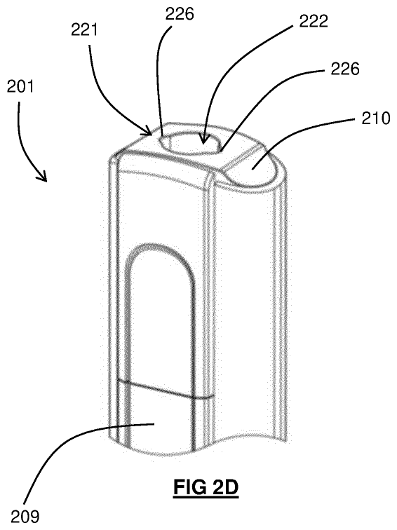

FIGS. 2A and 2B illustrate a heated-tobacco (HT) aerosol delivery system 200. The system 200 is an example of the system 100′ described in relation to FIG. 1B. System 200 includes an HT device 201 and an HT consumable component 202.

The device 201 and the consumable component 202 are configured such that the consumable component 202 can be engaged with the device 201. FIG. 2A shows the device 201 and the consumable component 202 in an engaged state, whilst FIG. 2B shows the device 201 and the consumable component 202 in a disengaged state.

The device 201 comprises a device body 209 and cap 210. In use the cap 210 is engaged at an end of the device body 209. Although not apparent from the figures, the cap 210 is moveable relative to the device body 209. In particular, the cap 210 is slideable and can slide along a longitudinal axis of the device body 209.

The device 201 comprises an output means (forming part of the UI of the device 201) in the form of a plurality of light-emitting diodes (LEDs) 211 arranged linearly along the longitudinal axis of the device 201 and on an outer surface of the body 209 of the device 201. A button 212 is also arranged on an outer surface of the body 209 of the device 201 and is axially spaced (i.e. along the longitudinal axis) from the plurality of LEDs 211.

FIG. 2C show a detailed section view of the consumable component 202 of the system 200. The consumable component 202 generally resembles a cigarette. In that respect, the consumable component 202 has a generally cylindrical form with a diameter of 7 mm and an axial length of 70 mm. The consumable component 202 comprises an aerosol forming substrate 213, a terminal filter element 214, an upstream filter element 215 and a spacer element 216. In other embodiments, the consumable may further comprise a cooling element. A cooling element may exchange heat with vapour that is formed by the aerosol-forming substrate 213 in order to cool the vapour so as to facilitate condensation of the vapour.

The aerosol-forming substrate 213 is substantially cylindrical and is located at an upstream end 217 of the consumable component 202, and comprises the aerosol former of the system 200. In that respect, the aerosol forming substrate 213 is configured to be heated by the device 201 to release a vapour. The released vapour is subsequently entrained in an airflow flowing through the aerosol-forming substrate 213. The airflow is produced by the action of the user drawing on a downstream 218 (i.e. terminal or mouth) end of the consumable component 202.

In the present embodiment, the aerosol forming substrate 213 comprises tobacco material that may, for example, include any suitable parts of the tobacco plant (e.g. leaves, stems, roots, bark, seeds and flowers). The tobacco may comprise one or more of leaf tobacco, stem tobacco, tobacco powder, tobacco dust, tobacco derivatives, expanded tobacco, homogenised tobacco, shredded tobacco, extruded tobacco, cut rag tobacco and/or reconstituted tobacco (e.g. slurry recon or paper recon). For example, the aerosol-forming substrate 213 may comprise a gathered sheet of homogenised (e.g. paper/slurry recon) tobacco or gathered shreds/strips formed from such a sheet.

In order to generate an aerosol, the aerosol forming substrate 213 comprises at least one volatile compound that is intended to be vaporised/aerosolised and that may provide the user with a recreational and/or medicinal effect when inhaled. The aerosol-forming substrate 213 may further comprise one or more additives. For example, such additives may be in the form of humectants (e.g. propylene glycol and/or vegetable glycerine), flavourants, fillers, aqueous/non-aqueous solvents and/or binders.

The terminal filter element 214 is also substantially cylindrical, and is located downstream of the aerosol forming substrate 213 at the downstream end 218 of the consumable component 202. The terminal filter element 214 is in the form of a hollow bore filter element having a bore 219 (e.g. for airflow) formed therethrough. The diameter of the bore 219 is 2 mm. The terminal filter element 214 is formed of a porous (e.g. monoacetate) filter material. As set forth above, the downstream end 218 of the consumable component 202 (i.e. where the terminal filter 214 is located) forms a mouthpiece portion of the consumable component 202 upon which the user draws. Airflow is drawn from the upstream end 217, thorough the components of the consumable component 202, and out of the downstream end 218. The airflow is driven by the user drawing on the downstream end 218 (i.e. the mouthpiece portion) of the consumable component 202.

The upstream filter element 215 is located axially adjacent to the aerosol-forming substrate 213, between the aerosol-forming substrate 213 and the terminal filter element 214. Like the terminal filter 214, the upstream filter element 215 is in the form of a hollow bore filter element, such that it has a bore 220 extending axially therethrough. In this way, the upstream filter 215 may act as an airflow restrictor. The upstream filter element 215 is formed of a porous (e.g. monoacetate) filter material. The bore 220 of the upstream filter element 215 has a larger diameter (3 mm) than the terminal filter element 214.

The spacer 216 is in the form of a cardboard tube, which defines a cavity or chamber between the upstream filter element 215 and the terminal filter element 214. The spacer 216 acts to allow both cooling and mixing of the vapour/aerosol from the aerosol-forming substrate 213. The spacer has an external diameter of 7 mm and an axial length of 14 mm.

Although not apparent from the figure, the aerosol-forming substrate 213, upstream filter 215 and spacer 216 are circumscribed by a paper wrapping layer. The terminal filter 214 is circumscribed by a tipping layer that also circumscribes a portion of the paper wrapping layer (so as to connect the terminal filter 214 to the remaining components of the consumable component 202). The upstream filter 215 and terminal filter 214 are circumscribed by further wrapping layers in the form of plug wraps.

Returning now to the device 201, FIG. 2D illustrates a detailed view of the end of the device 201 that is configured to engage with the consumable component 202. The cap 210 of the device 201 includes an opening 221 to an internal cavity 222 defined by the cap 210. The opening 221 and the cavity 222 are formed so as to receive at least a portion of the consumable component 202. During engagement of the consumable component 202 with the device 201, a portion of the consumable component 202 is received through the opening 221 and into the cavity 222. After engagement (see FIG. 2B), the downstream end 218 of the consumable component 202 protrudes from the opening 221 and thus also protrudes from the device 201. The opening 221 includes laterally disposed notches 226. When a consumable component 202 is received in the opening 221, these notches 226 remain open and could, for example, be used for retaining a cover in order to cover the end of the device 201.

Although not shown, the device body 209 comprises an electronics cavity. A power source, in the form of a rechargeable battery (a lithium ion battery), is located in electronics cavity.

The device 201 includes a connector (i.e. forming part of an IO module of the device 201) in the form of a USB port. The connector may alternatively be, for example, a micro-USB port or a USB-C port for examples. The USB port may be used to recharge the rechargeable battery.

The device 201 includes a controller (not shown) located in the electronics cavity. The controller comprises a microcontroller mounted on a printed circuit board (PCB). The USB port is also connected to the controller (i.e. connected to the PCB and microcontroller).

The controller is configured to control at least one function of the device 202. For example, the controller is configured to control the operation of the IR heater 204. Such control of the operation of the IR heater 204 may be accomplished by the controller toggling the electrical connection of the rechargeable battery 205 to the IR heater 204. For example, the controller is configured to control the heater 204 in response to a user depressing the button 212. Depressing the button 212 may cause the controller to allow a voltage (from the rechargeable battery 205) to be applied to the IR heater 204 (so as to cause the heating element to be heated).

The controller is also configured to control the LEDs 211 in response to (e.g. a detected) a condition of the device 201 or the consumable component 202. For example, the controller may control the LEDs to indicate whether the device 201 is in an on state or an off state (e.g. one or more of the LEDs may be illuminated by the controller when the device is in an on state).

The device 201 comprises a further input means (i.e. in addition to the button 212) in the form of a puff sensor. The puff sensor is configured to detect a user drawing (i.e. inhaling) at the downstream end 218 of the consumable component 202. The puff sensor may, for example, be in the form of a pressure sensor, flowmeter or a microphone. The puff sensor is operatively connected to the controller in the electronics cavity, such that a signal from the puff sensor, indicative of a puff state (i.e. drawing or not drawing), forms an input to the controller (and can thus be responded to by the controller).

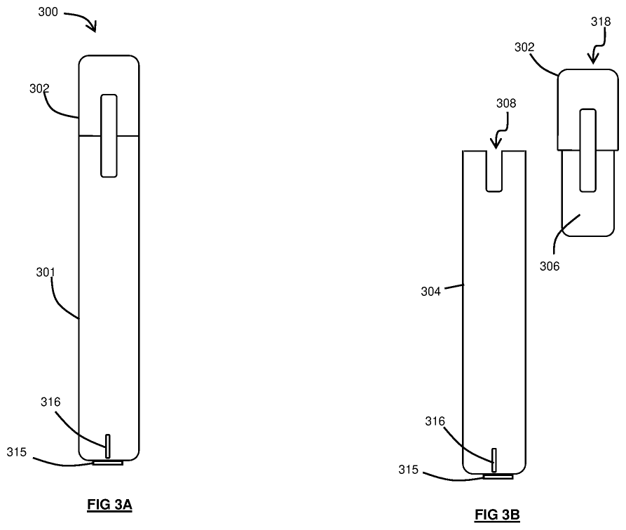

FIGS. 3A and 3B illustrate a vaping aerosol delivery system 300. The system 300 is an example of the systems 100 of FIGS. 1A and comprises a vaping device 301 and vaping component 302 (e.g. a vaping consumable component).

The device 301 and the component 302 are configured such that the component 302 can be engaged with the device 301. FIG. 3A shows the device 301 and the component 302 in an engaged state, whilst FIG. 3B shows the device 301 and the component 302 in a disengaged state. During engagement a portion of the component 302 is received in a recess 308 of the device 301. The component 302 is retained in the device 301 via an interference fit (although in other embodiments, the device and component could be engaged by screwing one onto (or onto) the other, through a bayonet fitting, or by way of a snap engagement mechanism).

The component 302 includes a tank 306. The tank 306 defines a reservoir for the storage of an aerosol- former, which in this embodiment, is in the form of e-liquid.

In this present embodiment, the component 302 is a “single-use” component. That is, upon exhausting the e-liquid in the tank 306, the intention is that the user disposes of the whole component 302. In other embodiments, the e-liquid (i.e. aerosol former) may be the only part of the system that is truly “single-use”. In such embodiments, the tank may be refillable with e-liquid or the e-liquid may be stored in a non-consumable component of the system. For example, the e-liquid may be stored in a tank located in the device or stored in another component that is itself not single-use (e.g. a refillable cartomizer).

In the illustrated system 300, an IR heater (not shown) is located in the component 302 and is configured to heat and vaporise the e-liquid (stored in the tank 306). Although not shown, the component comprises a porous wick. The porous wick conveys e-liquid from the tank 306 to a vaporising chamber which is a cavity within the component 302. Heat is transferred from the heating element to the e-liquid conveyed by the wick. This transfer of heat vaporises the e-liquid and the resultant vapour is entrained in an airflow passing through the component 302 (i.e. driven by a user drawing on a downstream end 318 of the component 302). Between the vaporisation chamber and the downstream end 318 (i.e. the mouth end), the vapour condenses into an aerosol, and is subsequently inhaled by the user.

Like the previously described embodiment, the device 301 comprises a power source in the form of a rechargeable battery (not shown) and a connector in the form of a USB port (not shown). The device 301 further comprises controller (also not shown). The rechargeable battery, connector and controller are similar (and operate in a similar manner) to the corresponding components of the embodiment described above with respect to FIG. 1A.

The component 302 includes a pair of heater electrical contacts 315 disposed on a device-facing end surface of the component 304. The heater electrical contacts 315 are electrically connected to the heater in the component 302, such that a voltage applied across the heater electrical contacts 315 generally corresponds to a voltage applied across the resistive heating element of the heater.

When the component 302 is engaged with the device 301, the heater electrical contacts 315 are brought into electrical contact with corresponding device electrical contacts (not shown) on the device 301. The device electrical contacts are electrically connected (directly or indirectly) to the rechargeable battery. The controller may thus be configured to control the voltage applied across the device electrical contacts from the rechargeable battery. By controlling the voltage applied across the device electrical contacts, the voltage applied to the heater is correspondingly controlled.

The device 301 includes an output means (forming part of the UI of the system 300) in the form of a single light-emitting diode (“LED”) 316. The LED 316 is operatively connected to the controller, such that controller can control the illumination of the LED 316. The controller is configured to illuminate the LED when then the heater is active.

The device 301 also includes an input means in the form of a puff sensor (not shown). The puff sensor is the same as that described above with respect to the embodiment shown in FIG. 1A.

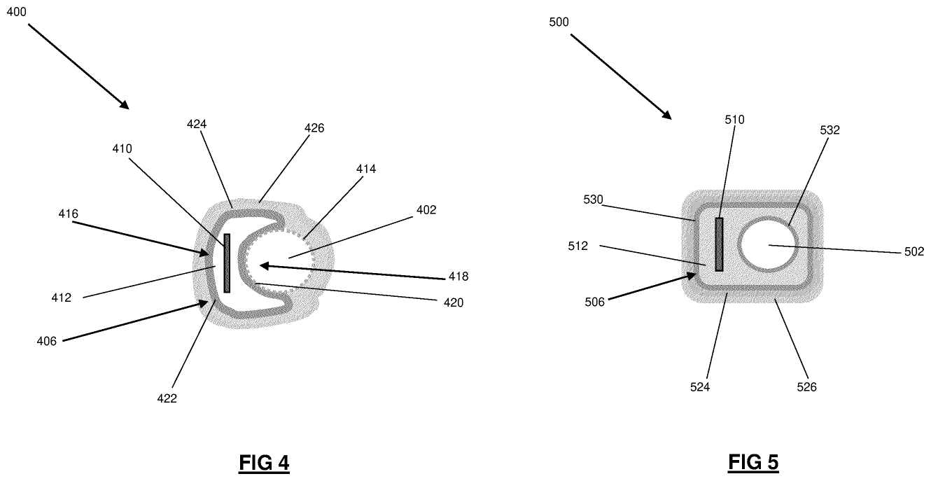

FIG. 4 illustrates a transverse cross-section through an article 400 comprising a cavity 402 for receiving the aerosol former and an infrared (IR) heater 406 in thermal communication with the cavity 402.

The IR heater 406 comprises a heating element 410 housed in a halogen-filled enclosure 412. The cavity 402 is defined by a chamber 414 and the enclosure 412 partly circumscribes the chamber 414. The chamber 414 is an elongate chamber with a circular transverse-profile i.e. the chamber is a cylindrical chamber. As shown in FIG. 4, the enclosure 412 comprises an outer wall 416, the outer wall 416 having an indented portion 418 that partly circumscribes the cavity 402. The indented portion 418 is in contact with the chamber 414. The indented portion 418 defines an arc projecting inwardly into the enclosure 412. The arc has an arc angle approximately equal to 180 degrees. The outer wall not forming the indented portion 418 also forms an arc having a greater arc radius than the indented portion 418, such that the enclosure 412 has a crescent-shaped transverse profile.

The enclosure 412 is formed of thermally conductive material (e.g. lime glass or Pyrex).

The heating element 410 is an electrode and is located so that only a minor portion of the perimeter of the cavity 402/chamber 414 (e.g. a portion subtending an angle of around 50 degrees) faces the heating element 410 such that the heating element 410 is on only one of the lateral sides of the cavity 402.

The article 400 comprises a reflector 424 that circumscribes both the IR heater 406 and the chamber 414. In particular, the reflector 424 is a metal coating, coating the outer wall of the enclosure 412 and the chamber 414.

The article 400 also comprises an insulator 426 which circumscribes the reflector 424.

FIG. 5 illustrates a transverse cross-section through another embodiment of an article 500 where the enclosure 512 completely circumscribes the cavity 502. In this embodiment, the enclosure 512 comprises an outer wall 530 and an inner wall 532, the outer wall 530 surrounding the inner wall 532 such that the enclosure 512 is defined therebetween. The inner wall 532 defines the cavity 502 and has a circular wall. The outer wall 530 has a transverse profile that is substantially rectangular and the axial centre of the profile define by the outer wall 530 is laterally offset from the axial centre of the cavity 502.

In the embodiment of FIG. 5, the reflector 524 fully circumscribes the outer wall 530 of the enclosure 512 and the insulator 526 fully circumscribes the reflector 524.

Again, the heating element 510 is an electrode and is located so that only a minor portion of the perimeter of the cavity 502/inner wall 532 (e.g. a portion subtending an angle of around 50 degrees) faces the heating element 510.

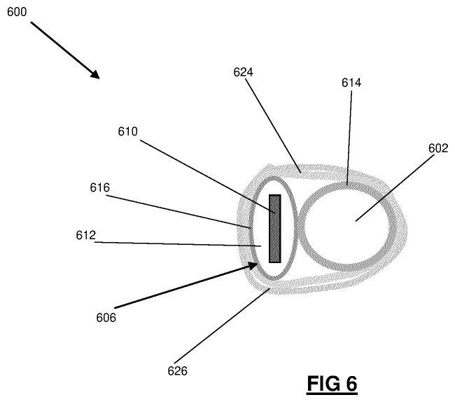

FIG. 6 illustrates a transverse cross-section through another embodiment of an article 600 where the cavity 602 is defined by a cylindrical chamber 614. IR heater 606 is located so that only a minor portion of the perimeter of the cavity 602/chamber 614 (e.g. a portion subtending an angle of around 50 degrees) faces the heating element 610 such that the heating element is on only one of the lateral sides of the cavity 602 and the reflector 624 and insulator 626 surround both the IR heater 606 and the chamber 614.

In some embodiments, the article 400, 500, 600 is a device (for example a HT device as described above in relation to FIGS. 2A and 2B), where the device is configured for engagement with a HT consumable. For example, the cavity 402, 502, 602 may be in communication with the opening 221 shown in FIG. 2D The IR heater 406, 506, 606 is configured to heat and vaporise the HT consumable (shown in FIG. 2C) such that the HT consumable releases at least one volatile component to form an aerosol. The device may otherwise include the features described in relation to the HT device of FIGS. 2A-2D.

In some embodiments, the article 400, 500, 600, is a component (for example a vaping component as described above in relation to FIGS. 3A and 3B) and configured for engagement with a device. In these embodiments, the IR heater is in the component. The component comprises a tank which defines a reservoir for storage of the aerosol former in the form of e-liquid. The component comprises a porous wick that draws e-liquid from the tank. The wick is disposed in a vaporising chamber which defines the cavity of the component for receiving the aerosol former (i.e. the e-liquid). When the heating element 410, 510, 610 is heated, heat is transferred from the heating element 410, 510, 610 through the enclosure 412, 512, 612 and to the e-liquid conveyed by the wick in the cavity/vaporising chamber. The component may otherwise include the features described in relation to the vaping component of FIGS. 3A and 3B.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the words “have”, “comprise”, and “include”, and variations such as “having”, “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means, for example, +/−10%.

The words “preferred” and “preferably” are used herein refer to embodiments of the invention that may provide certain benefits under some circumstances. It is to be appreciated, however, that other embodiments may also be preferred under the same or different circumstances. The recitation of one or more preferred embodiments therefore does not mean or imply that other embodiments are not useful, and is not intended to exclude other embodiments from the scope of the disclosure, or from the scope of the claims.

1. An aerosol delivery article (400) comprising:
a cavity (402, 502) for receiving an aerosol former; and
an infrared heater (406, 506) in thermal communication with the cavity (402, 502), the infrared heater (406, 506) comprising at least one heating element (410, 510) in a halogen-filled enclosure (412, 512) wherein the enclosure (412, 512) at least partly circumscribes the cavity (402, 502); characterised in that the cavity (402, 502) has a perimeter and a major portion of the perimeter faces away from the heating element(s) (410, 510), such that a line extending from an axial centre of the cavity (402, 502) through said major portion of the perimeter does not intersect the heating element(s) (410, 510).

2. (canceled)

3. (canceled)

4. An article (400) according to claim 1 wherein the major portion of the perimeter subtends an angle of equal to or greater than 270 degrees.

5. (canceled)

6. An article (400) according to claim 1 wherein the cavity (402, 502) is at least partly defined by the enclosure (412, 512).

7. An article (400) according to claim 1 wherein the cavity (402, 502) is defined by a chamber (414) and the enclosure (412, 512) at least partly circumscribes the chamber (414).

8. An article (400) according to claim 7 wherein there is direct contact between the enclosure (412, 512) and the chamber (414).

9. (canceled)

10. An article (400) according to claims 1 wherein the enclosure (412) comprises an outer wall (416), the outer wall (416) having an indented portion, the indented portion at least partly circumscribing the cavity (402).

11. An article (400) according to claim 10 wherein the indented portion defines an arc projecting inwardly into the enclosure (412), the arc having an arc angle equal to or greater than 180 degrees.

12. An article (400) according to claim 1, wherein the enclosure (512) comprises an outer wall (530) and an inner wall (532), the outer wall surrounding the inner wall with the enclosure (512) defined therebetween, wherein the inner wall (532) circumscribes the cavity (502).

13. An article (400) according to claim 12 wherein the axial centre of a profile defined by the outer (530) wall is laterally offset from the axial centre of the cavity (502)/chamber.

14. An article (400) according to claim 1 wherein the article (400) is an aerosol delivery device configured for engagement with a heat not burn aerosol delivery component comprising the aerosol former.

15. An article (400) according to claim 1 wherein the article (400) is an aerosol delivery component comprising the aerosol former configured for engagement with a vaping device.
